(19) United States
(12) Patent Application Publication
(10) Pub. No.: US 2004/0254587 A1
(43) Pub. Date: Dec. 16, 2004

(54) Pedicure implement having a contoured surface

(76) Inventor: Joon Park, Glendale, CA (US)

Correspondence Address: CHRISTIE, PARKER & HALE, LLP, PO BOX 7068, PASADENA, CA, 91109-7068, US

(21) Appl. No.: 10/489,235

(22) PCT Filed: Sep. 12, 2002
(86) PCT No.: PCT/US02/28939

Related U.S. Application Data
(60) Provisional application No. 60/318,408, filed on Sep. 12, 2001.
(60) Provisional application No. 60/351,229, filed on Jan. 25, 2002.

(51) Int. Cl. A61B 17/50

(52) U.S. Cl. 606/131

(57) ABSTRACT

A pedicure implement for removing human foot callus and corn is provided. The pedicure implement has a body made up of at least two surfaces, which meet at a circumferential rim area. At least one of the surfaces has distinctively concave and convex areas, and at least one of the surfaces is abrasive at its external surface. The rim area is not abrasive at its external surface so as to reduce a change for cutting a skin. A pedicure implement may be mounted on a motorized appliance to generate motion so as to remove callus and corn from human foot.

### CROSS REFERENCE TO RELATED APPLICATION(S)

This application fully incorporates by reference the contents of U.S. Provisional Application No. 60/318,408 entitled “Fresh Foot Care” filed Sep. 12, 2001 and U.S. Provisional Application No. 60/351,229 entitled “Fresh Foot Care” filed Jan. 25, 2002. This application contains subject matter related to the subject matter disclosed in U.S. Pat. No. 6,165,059 entitled “Abrasive Medium with Selected Density,” the contents of which are fully incorporated by reference herein.

### FIELD OF THE NVENTION

This invention is related to a pedicure implement, and particularly to a hand-held pedicure implement that is flexible, floatable, and contoured for convenient use, of which abrasive surfaces provide capabilities for removing unwanted skin.

### BACKGROUND OF THE INVENTION

Pedicure products are typically used for the removal of rough, dead, or excess skin, such as calluses and corns, which accumulate under human feet. These pedicure products typically have abrasive surfaces that help to smooth rough skin.

Recent pedicure products have included devices usable in the shower or tub. However, these devices typically having an abrasive grit applied on one end and a handle device disposed on the other end, are generally heavy enough to sink in water. Some of the other pedicure products for use in shower or tub available in the market include pumice and Sanding Sponge manufactured by 3M®, part number 909NA or CP-O4ONA, which is constructed as a generally rectangular block with abrasive grits on four adjoining flat surfaces and without grits on the other two side surfaces. The substrate of this Sanding Sponge is an open cell sponge, which is subject to absorb water/human skin debris, which may lead to fungus or bacteria buildup. 3M ® is a registered trademark of Minnesota Mining and Manufacturing Company, Saint Paul, Minn. Although natural or artificial pumice floats in water, the characteristics of open pores and deep cavities present the problem of fungi and bacteria that are generated and possibly transmitted to others through use.

U.S. Pat. No. 6,178,970 (“Purifoy et al.”) discloses a motorized personal grooming sander device with a sheet of sandpaper attached thereon to remove calluses and other aberrant cellular growth from the feet. The potential problems with this sander device include that the flat sandpaper may fail to reach the middle bottom section, or concave areas, of the feet, and that the torque generated by the motor may dislocate the grooming sander device away from the feet.

Some pedicure products have been made of hard materials, such as sandpaper on hard plastic or stone, which do not conform to the contours of the feet. The hardness contributes not only to the factor of discomfort, but also to more time and effort that is required to rub the foot. Further, different degrees of roughness may be required to efficiently remove different corns/calluses having different degrees of hardness.

Therefore, it is desirable to provide a pedicure product with flexibility to remove callous skin from feet in a comfortable and efficient manner with one or more of the following features: 1) an ability to float in water; 2) at least two different degrees of roughness; 3) reduced likelihood of bacteria/fungus buildup; and 4) contoured (convex and/or concave) surfaces.

### SUMMARY OF THE INVENTION

In an exemplary embodiment according to the present invention, a pedicure implement for removing human foot callus and corn is provided. The pedicure implement includes a body having at least two surfaces, wherein said surfaces meet at a circumferential rim area, at least one of said surfaces having distinctively concave and convex areas, and at least one of said surfaces is abrasive at its external surface, wherein said rim area is not abrasive at its external surface so as to reduce a chance for cutting a skin.

In another exemplary embodiment according to the present invention, a pedicure implement for removing human foot callus and corn is provided. The pedicure implement includes a body comprising at least two surfaces, wherein said at least two surfaces are formed of one piece, at least one of said surfaces having distinctly concave and convex areas, and at least one of said surfaces is abrasive at its external surface.

In yet another exemplary embodiment according to the present invention, a pedicure implement for removing human foot callus and corn is provided. The pedicure implement includes a body comprising two surfaces, wherein said surfaces meet at a circumferential rim area, one of said surface is a concave surface and the other said surface is a convex surface, at least one of said surfaces is abrasive at its external surface, wherein said body has a density less than that of water.

In still another exemplary embodiment according to the present invention, a motorized pedicure implement is provided. The motorized pedicure implement includes a body having a first end and a second end; and a motion head disposed on said first end of the body, wherein the motion head comprising three motorized rotating areas capable of receiving removable disks, said disks being suitable for treating calluses on feet, said disks are abrasive at their respective external surfaces, and said disks are positioned in a manner to allow for torque neutralizing.

In a further exemplary embodiment according to the present invention, a motorized pedicure implement is provided. The motorized pedicure implement includes a body having a first end and a second end; and a motion head disposed on said first end of the body, wherein the motion head comprises a motorized rotating area capable of receiving a removable device, said device being suitable for treating calluses on the feet, wherein said device is abrasive at an external surface, and said device has a general shape of a bowling pin that has convex and concave areas in a longitudinal direction.

### BRIEF DESCRIPTION OF THE DRAWINGS

These and other aspects of the invention may be understood by reference to the following detailed description, taken in conjunction with the accompanying drawings, wherein:

FIG. 1 is a top view of a first exemplary embodiment 10 of the pedicure implement in accordance with the present invention;

FIG. 2 is a front view of the first exemplary embodiment 10 of FIG. 1;

FIG. 3A is a cross-sectional view taken along line 3-3 of FIG. 1 having a foam and sealed interior;

FIG. 3B is a cross-sectional view taken along line 3-3 of FIG. 1 with a hollow interior;

FIG. 4 is a front view of a second exemplary embodiment 14 of a pedicure implement in accordance with the present invention;

FIG. 5 is a side view of the second exemplary embodiment 14 of FIG. 4;

FIG. 6A is a cross-sectional view taken along line 6 of FIG. 4 with an elastic abrasive pad mounted on a support;

FIG. 6B is a cross-sectional view taken along line 6 of FIG. 4 with a metallic abrasive pad mounted on a support; and

FIG. 7 is a partial cross-sectional view for an abrasive assembly, generally a conical shape, mounted on a support.

### DESCRIPTION OF THE EMBODIMENT

The pedicure implement in exemplary embodiments according to the present invention provides for smoothing rough skin, dead skin, skin build-up, calluses, and the like on human feet. The pedicure implement may be light and floatable, with a density less than that of water, and may be flexible and/or be convex/concave to comfortably reach contours of the feet. The pedicure implement is resistant to fungus buildup and mildew, resulting from being non-porous, and may have different grit selected for the upper and lower surfaces. The pedicure implement may have selected abrasive areas and non-abrasive areas to increase the effectiveness of abrading, and may have dyed color tones applied to distinguish one surface from another, which has a different distribution of grit.

In other exemplary embodiments according to the present invention, a motorized pedicure implement with multiple abrading disks to counterbalance the torque generated by the motor may be provided. The motorized pedicure implement may have a compliance mechanism for abrading effectiveness. The motorized pedicure implement may have pliable, convex/concave and/or conical abrading pads to reach every part of the feet.

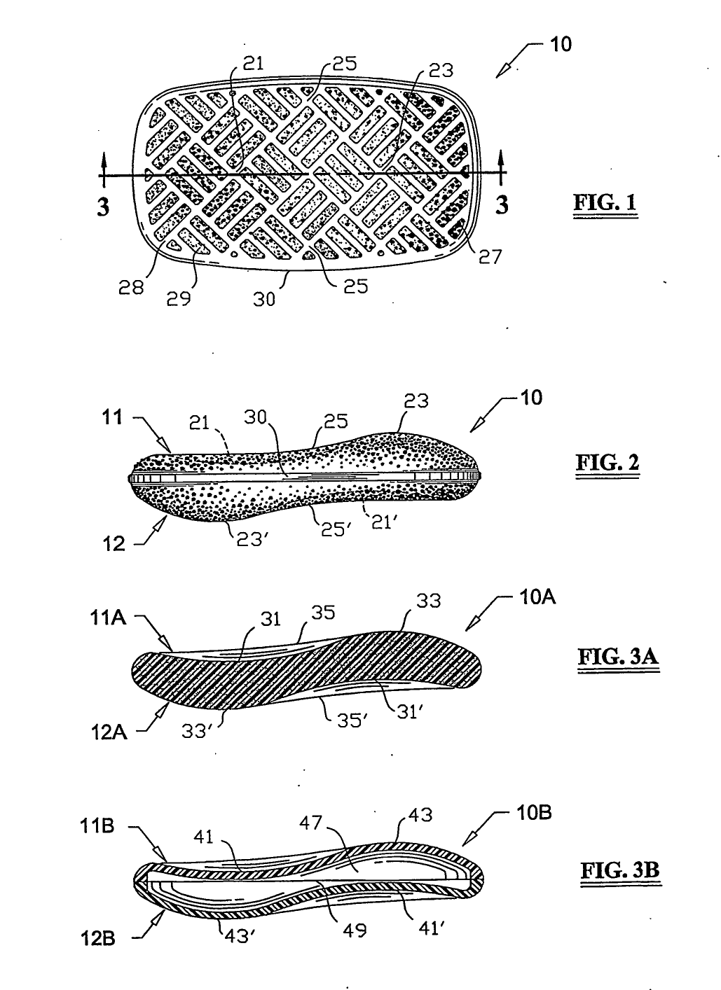

A pedicure implement 10 (or a body thereof) in a first exemplary embodiment according to the present invention is illustrated as top and front views in FIGS. 1 and 2, respectively. FIG. 1 shows a top view surface of an upper half 11 and FIG. 2 shows a side view of the upper half 11 and a lower half 12. The pedicure implement 10 has a generally rectangular box shape with rounded corners and a contour shape as shown in FIGS. 1 and 2. Concave areas 21 and 21′ and convex areas 23 and 23′ with two ridges 25 and 25′ form the upper half 11 and the lower half 12, respectively. The upper half 11 is configured substantially identically to the lower half 12 in the first exemplary embodiment. In other embodiments, the upper and lower halves may be configured differently from one another. The upper and lower halves may also be referred to herein as top and bottom halves, respectively.

The external surfaces of the upper half 11 and the lower half 12 may be made abrasive by making the external surfaces themselves abrasive. The external surfaces of the upper and lower halves may also be made abrasive by treating them with abrasives, which may have different grain/grit sizes to provide desired roughness. Similarly, all abrasive surfaces as disclosed herein may be made abrasive through making the external surfaces themselves abrasive and/or applying/treating with abrasives.

As shown in the cross-sectional view in FIG. 3A, a pedicure implement 10A is constructed of foam material. The pedicure implement 10A has a unibody, which means that the pedicure implement I OA is formed from a single piece of material. FIG. 3A illustrates a top half 11A and a bottom half 12A with concave areas 31 and 31′ and convex areas 33 and 33′ with two ridges 35 and 35′, respectively. In other embodiments, the pedicure implement 10A may be constructed from any other suitable material.

For example, the pedicure implement 10A may be manufactured with PVC, neoprene, polyethylene, or urethane foams and by any of structural-foam molding, foam molding, and prefabricated foams. Using these materials and fabrication methods, the joining may not be needed. However, the materials for the pedicure implement 10A should be of a closed-cell foam, because the open-cell foam is subject to absorbing water/human skin debris, which may cause fungus and/or bacteria buildup, and may transmit diseases from other users. Since closed-cell foam materials provide multiple air pockets, the pedicure implement 10A with the abrasive grit disposed thereon can be made to float on water. Hence, the pedicure implement 10A may be viewed as having a density less than that of water.

In FIG. 3B, a pedicure implement 10B has a shell body formed of an upper shell 11B and a lower shell 12B, which define and enclose a hollow space 47. The halves 11B and 12B have convex areas 43 and 43′, respectively, and concave areas 41 and 41′, respectively. The shell body may be a unibody constructed from a single piece of material, or it may be constructed with two halves, which are attached and sealed around adjoining rims 49. In other embodiments, the pedicure implement 10B may comprise a shell body made from more than two pieces of same or different materials. In other embodiments, an inexpensive pedicure implement 10B may comprise only one shell 1lB with multiple ribs that are disposed to extend from inner surfaces of the concave area 41 and the convex area 43. Since the pedicure implement 10B, whether it is formed of a unibody or of two shells, have a hollow interior, it floats in water with a proper choice of material. Hence, it may be viewed as having an average density less than that of water.

In order to prevent excessive deflection when force is applied, the pedicure implement 10B may include one or more cylindrical shapes or supports that are disposed to extend from inner surfaces of the concave areas 41 and 41′ into the hollow interior, and towards the inner surfaces of the convex areas 43 and 43′, respectively. A tongue and groove may be utilized in the adjoining rims 49 to tightly hold the halves 11B and 12B.

The shell body of the pedicure implement 10B of FIG. 3B may be manufactured with elastomers such as elastic urethane, polyester, polypropylene, PVC, ABS, K-Resin and any thin polymers that provide elasticity. The hollow interior 47 provides buoyancy in water. The shell body of the pedicure implement 10B may be manufactured using any of blow molding, twin-sheet thermoforming, gas-assist injection molding, and rotational molding, all of which provide a hollow interior 47 without any adjoining rim. Further, ceramic material may be used to fabricate the shell body for the pedicure implement 10B. Even when the shell body of the pedicure implement 10B is a unibody (i.e., made of a single piece), and therefore without any adjoining rim, the circumferential rim area may be defined at a location where the upper (concave and convex) surface meets the lower (concave and convex) surface.

In other embodiments, each shell 11B and 12B may be made with two injection molded or thermoformed parts that are joined at their adjoining rims 49 by a resin, the ultrasonic welding process, or thermal means. In still other embodiments, one of the shells 11B and 12B may be replaced with a brush assembly, while the other one remains as a shell as described above. The shell body 10B provided with a hollow interior 47 can be made to float on water.

Even though the embodiments of FIGS. 1-3B have been generally referred together as the first embodiment herein, they may actually represent at least three distinct embodiments as follows: 1) unibody made of a foam material; 2) unibody made, for example, of elastomer or polymer material having a hollow interior; and 3) two shells made, for example, of elastomer or polymer material having a hollow interior. As can be seen on FIG. 2, the circumferential rim area 30 in each of these embodiments may not be abrasive at its external surface so as to reduce a chance for cutting a slin.

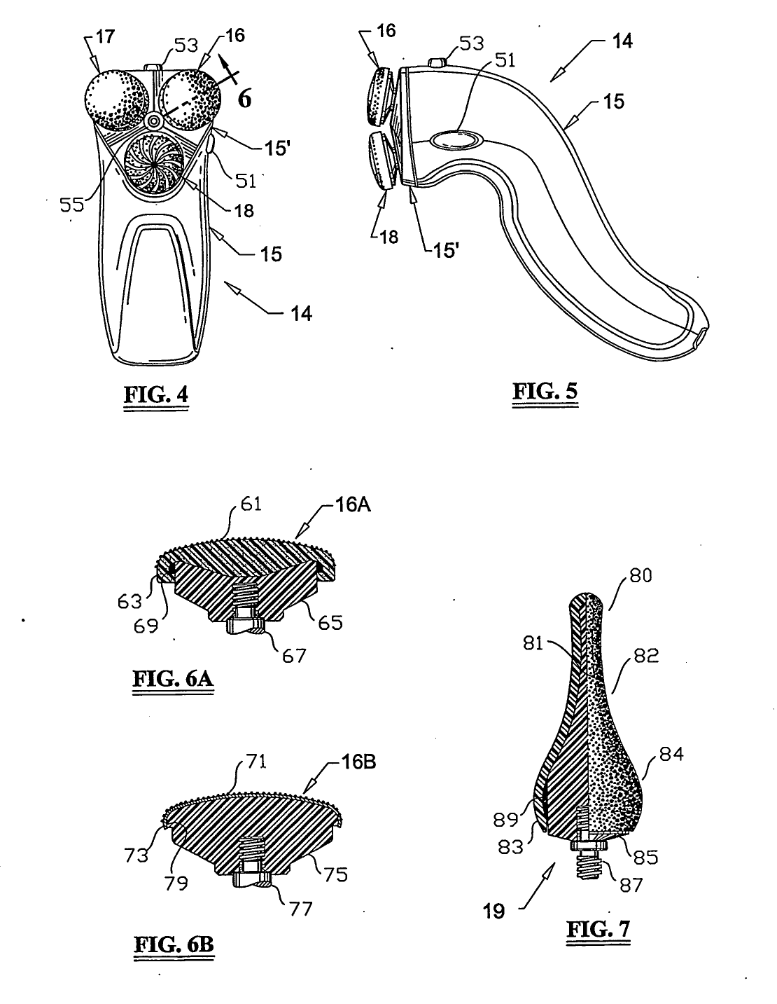

In a second exemplary embodiment according to the present invention, a motorized pedicure implement 14 is provided. The front and side views of the motorized pedicure implement 14 are illustrated in FIGS. 4 and 5, respectively. The motorized pedicure 14 includes housing 15, which is an ergonomically shaped case that contains a motor, gearing and a switch 51. The housing 15 may also contain a rechargeable battery to supply power to the motor. For embodiments where the motorized pedicure implement 14 may be used during shower or bath, the motor should be supplied with power from a battery rather than an electrical outlet.

The exemplary motorized pedicure implement 14 includes three abrasive pad assemblies 16, 17 and 18 disposed at one end of housing 15 on head assembly 15′ near the switch 51. These abrasive pad assemblies 16, 17 and 18 provide rotary or orbital abrading motion generated by the motor embedded inside of the housing 15. In other embodiments, more or less than three abrasive pad assemblies may be provided on the motorized pedicure implement. Alternatively, the motor may drive a rotating shaft 55 with a thread (which may be female), which is located at approximately the center of the pad assemblies 16, 17 and 18. The rotating shaft 55 may be used with a singular abrasive pad assembly, which for example, may be selected from abrasive pad assemblies 16A, 16B, and 19 as illustrated on FIGS. 6A, 6B and 7, respectively. The motorized pedicure 14 also includes a lock 53, which may be pressed to change the singular abrasive pad assembly mounted on the rotating shaft 55.

Each of a synthetic pad 63 of the abrasive pad assembly 16A and a metallic pad 73 of the abrasive pad assembly 16B has a generally convex shape and is mounted on supports 65 and 75, respectively. In turn, the supports 65 and 75 are fastened to rotating shafts 67 and 77, respectively, to form pad assemblies 16A and 16B, as shown in FIGS. 6A and 6B, respectively. As seen in FIG. 6A, an inward flange 69 may be used to secure the synthetic pad 63 to the support 65 in the abrasive pad assembly 16A. Similarly, the metallic pad 73 may be secured by radially inward swage 79 onto the support 75, as shown in FIG. 6B. Further, synthetic pad 63 may also have radially disposed teeth 69 to help secure the pad to the support 65 by interlocking with radially disposed teeth on the support 65.

FIG. 7 illustrates an abrasive cone assembly 19 whose cross section is a generally conical shape. As can be seen in FIG. 7, the basic structure of the abrasive cone assembly 19 is similar to that of the abrasive pad assembly 16A of FIG. 6A. The shape of the abrasive cone assembly 19 for the pedicure application may include a cylinder that is at least ½ inch in diameter and 1 inch in length, as well as a bowling pin shape as shown. Of course, the dimensions of the cone assembly may be different in other embodiments.

Elastomer and foam materials as discussed above in reference to FIGS. 3A and 3B may be used to fabricate the synthetic pads 63 and 83 in FIGS. 6A and 7, respectively. Adhesive and grit may be bonded directly onto the adhesive friendly polymers, including ABS, PVC, or urethane, to replace two materials that are used for the support 67 and the synthetic pad 63 in FIG. 6A. Another method to adhere the grit onto the substrate is by depositing highly heated grit onto the substrates that are made of polymer material. The cone abrasive assembly 19 may be constructed using the same method as well. Another material for the metallic pad 73 in FIG. 6B may be sheet metal, including hardened stainless steel with a rough surface or with abrasive grit bonded thereon.

To generate roughness on the surfaces of the pedicure implement 10 and the pad assemblies 16, 16A, 16B, 17, 18 and 19, various economical media such as aluminum oxide, silicon carbide, garnet, pumice, or silica can be applied on the entire or selected areas by resin bond, such as polyurethane adhesive on the upper halves 11 and lower halves 12 and on the synthetic pads 63 and 83. The process of bonding grit 27 on the surface of the halves 11 and 12, grit 61 on the synthetic pad 63, and grit 81 on the synthetic pad 83 is similar to the sandpaper manufacturing process, but the adhesive and substrate material are to withstand soap and strong cleaning fluid use, as well as boiling water temperatires for sterilization. The roughness may also be provided to the surfaces of the pedicure implement 10 and the pad assemblies 16, 16A, 16B, 17, 18 and 19 by making the external surfaces themselves abrasive.

The grit 71, as shown in FIG. 6B, may be formed from any hard material including diamond, tungsten carbide, silicon carbide, boron carbide and/or cubic boron nitride. It can be deposited on the metallic pad 73 by the methods including flame spraying, plasma arc spraying, or brazing. The metallic pad 73 can withstand wear against abrading long enough even with aluminum material with hard anodizing followed by rough surface texture.

Due to the two major areas that provide convex and concave areas on each of the halves 11 and 12 in FIG. 3, it would be sensible to provide a different roughness on each half or area with a different color for easier identification. For example, the upper half 11 of FIG. 2 may be colored lavender and provided with a grit size between 36 and 80, while the lower half 12 may be colored blue and provided with a grit size between 60 and 120. Of course, colors other than lavender and blue may be selected for the halves to distinguish between the halves easier. Further, grit sizes on the upper and lower surfaces may be different in other embodiments.

The pedicure implement 10A may be manufactured with a flat foam sheet whose two surfaces are treated with different size grits and different color adhesives followed by thermoforming and trimming to bring the body shape as illustrated in FIGS. 1-3A.

Due to the rotating abrading provided by the motor, the abrasive grit 61 in FIG. 6A, the grit 71 in FIG. 6B, and the grit 81 in FIG. 7 should have a finer grit size within the range of 60 to 320.

The recommended use of the pedicure implement 10 involves the application to the feet when the feet are wet. Removing dead skin, calluses, corns, and the like is achieved when applying the device to the feet in any convenient rubbing motion. The specific shape of the body 10 allows the user to easily grip the device in either hand while the exposed surface of the device is ready for use. Compatible with the contours of the feet, the upper and lower surfaces of the body 10 have convex areas 23 and 23′ for the middle bottom section of the feet as well as concave areas 21 and 21′ for all other convex areas of the feet. The convex and concave areas 23, 23′, 21, and 21′ can come into contact with the feet, particularly with the sole of the feet. The contoured features and flexibility of body 10 allow the user to effectively apply pressure, as it fits strategically with the natural shape of the feet, for the purpose of removing undesired skin with comfort and effectiveness. Additionally, the upper half 11 can have a different roughness from the lower half 12 so that the use of the rough half can be followed by that of the smoother half. In other embodiments, the top half 11 may be entirely convex while the bottom half 12 may be entirely concave.

Additional effectiveness may be achieved by providing selected abrasive areas 29 and non-abrasive areas 28 as illustrated in FIG. 1. Furthermore, the rim 30 and its adjoining area in FIGS. 1 and 2 should not be abrasives since these non-contoured edges with abrasives may cut non-callous skin.

The motorized pedicure implement 14 provides removal of rough skin, dead skin, skin build-up, calluses, and the like on human feet more effectively by means of electricity. The arrangement of these three rotating/abrading pad assemblies 16, 17 and 18 counterbalances the torque generated by the motor. Furthermore, each abrading pad assembly 16, 17 or 18 may have a center compliance mechanism including a compression spring, which is located between the end of shaft 67 and the output shaft that is at the end of the gear/motor. This center compliance mechanism will conform to the contours on different areas of the human feet.

The central area among the three assemblies 16, 17 and 18 may be effectively applied to the convex area of the feet and any one or two pad assemblies may reach the concave area or middle area of the feet. Furthermore, these pad assemblies may be easily removed for the user to select a different grit size pad assembly using a male thread on the end of shaft 67 or 77 with a female thread on the center of support 65 and 75, as shown as an example in FIGS. 6A and 6B, respectively.

A spiral rib works as abrasive and non-abrasive channels, which serve as exits for the removed dead skins, as shown as an abrasive pad assembly 18 in FIG. 4.

Yet another convenient way to change to different roughnesses is to change the whole head assembly 15′, which holds three pad assemblies 16, 17 and 18. The head assembly 15′, 30 for example, may be mounted or separated to and from the front end of the housing 15 by a snap.

The rotating abrasive cone assembly 19 may be very effective to be used on almost any part of the feet: a narrow cone tip 80 for the area between the toes, a cone middle 82 that is concave shape for the convex area of the foot, and a cone bottom 84 that is convex shape for the concave area of the feet, all as illustrated in FIG. 7.

The functions of the motorized pedicure implement 14 may be further extended to the combined motion of rotation and oscillation, or oscillatory motion only by the manipulation of cam and gearing mechanism embedded inside the housing 15. This way, the effectiveness of the removal of unwanted skin and of massage may be increased. For example, for the combined motion of rotation and oscillation provided by the motorized pedicure implement 14, the abrasive cone assembly 19 may be used as described previously. For the oscillatory motion, however, the abrasive assembly 19 may be modified to a flat bar similar to a file with two major surfaces, a triangular bar with three surfaces, or even a square bar with four surfaces. Each surface may be relatively flat and/or convex/concave and with different abrasives and dye applied onto them.

Slight modification of conventional linear/orbital action electrical pad sanders can be useful for the removal of calluses and corns from human feet, wherein the bottom surface of the sander is provided with a concave/convex area with abrasive lower shell 12B as shown in FIG. 3B and the upper shell 11B is modified and detachably mounted onto the lower end of the sander to generate motion so as to remove calluses and corns.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come within the meaning and range of equivalents thereof are intended to be embraced therein.

1. A pedicure implement for removing human foot callus and corn comprising:
a body comprising at least two surfaces, wherein said surfaces meet at a circumferential rim area, at least one of said surfaces having distinctively concave and convex areas, and at least one of said surfaces is abrasive at its external surface,
wherein said rim area is not abrasive at its external surface so as to reduce a chance for cutting a skin.

2. The pedicure implement of claim 1, wherein the abrasiveness is provided by at least one material selected from a group consisting of aluminum oxide, silicon carbide, garnet, pumice, tungsten carbide, diamond and silica.

3. The pedicure implement of claim 2, wherein said at least two surfaces are abrasive at their respective external surfaces.

4. The pedicure implement of claim 3, wherein roughness of a first abrasive applied to one of said two surfaces is different from the roughness of a second abrasive applied to the other of said two surfaces, wherein the first and second abrasives provide the abrasiveness of the respective external surfaces.

5. The pedicure implement of claim 3, wherein color applied to one of said at least two surfaces is different from the color applied to another of said at least two surfaces.

6. The pedicure implement of claim 1, wherein said body is generally rectangular shaped, and has rounded corners.

7. (Canceled)

8. The pedicure implement of claim 1, wherein said body has a hollow interior.

9. The pedicure implement of claim 1, wherein said body has a density less than that of water.

10. The pedicure implement of claim 1, wherein said abrasive external surface comprises at least one of abrasive areas and non-abrasive areas, and raised abrasive areas and lowered abrasive areas, whereby an effectiveness of abrading is increased.

11. The pedicure implement of claim 1, wherein one of said at least two surfaces is abrasive at its external surface and an opposing one of said surfaces has a brush disposed thereon.

12. The pedicure implement of claim 1, wherein one of said at least two surfaces is abrasive at its external surface and an opposing one of said surfaces is configured to be detachably mounted onto a motorized appliance to generate motion so as to remove said callus and corn from human foot.

13. A pedicure implement for removing human foot callus and corn comprising:
a body comprising at least two surfaces, wherein said at least two surfaces are formed of one piece, at least one of said surfaces having distinctly concave and convex areas, and at least one of said surfaces is abrasive at its external surface.

14. The pedicure implement of claim 13, wherein said body comprises at least one material selected from a group consisting of sheet metal, thermoplastic, elastomer, foam material, ceramic, and pumice stone.

15. A pedicure implement for removing human foot callus and corn, said pedicure implement comprising:
a body comprising two surfaces, wherein said surfaces meet at a circumferential rim area, one of said surface is a concave surface and the other said surface is a convex surface, at least one of said surfaces is abrasive at its external surface, wherein said body has a density less than that of water.

16. A motorized pedicure implement comprising:
a body having a first end and a second end; and
a motion head disposed on said first end of the body,
wherein the motion head comprises three motorized rotating areas capable of receiving removable disks, said disks being suitable for treating calluses on feet, said disks are abrasive at their respective external surfaces, and said disks are positioned to provide at least one of torque neutralizing, and reaching the concave and convex areas of the feet.

17. The pedicure implement of claim 16, wherein at least one of said disks is convex shaped.

18. The pedicure implement of claim 16, wherein at least one of said disks has non-abrasive channels through which removed dead skin can exit.

19. The pedicure implement of claim 16, wherein the body is engineered to fit a shape of a user's palm.

20. A motorized pedicure implement comprising:
a body having a first end and a second end; and
a motion head disposed on said first end of the body,
wherein the motion head comprises a motorized movement area capable of receiving a removable device, said device being suitable for treating calluses on a foot,
wherein said device is abrasive at an external surface, and has convex and concave areas.

21. The pedicure implement of claim 20, wherein the body is engineered to fit a shape of a user's palm.

22. The pedicure implement of claim 20, wherein said movement is a rotary motion, and wherein said device has a general shape of a bowling pin.

23. The pedicure implement of claim 20, wherein said movement comprises a linear motion and an orbital motion, and wherein said device is generally rectangular shaped with rounded corners.

24. The pedicure implement of claim 1, wherein said rim area is constructed as a separate structure from that of said two surfaces.
